(19) United States
(12) Patent Application Publication
(10) Pub. No.: US 2013/0345655 A1
(43) Pub. Date: Dec. 26, 2013

(54) ENZYME-POLYMER MATRIX BEADS FOR DE-ODOR APPLICATIONS

(71) Applicants: Songtao Wu, Ann Arbor, MI (US); Hongfei Jia, Ann Arbor, MI (US); Masahiko Ishii, Okazaki City (JP); Minjuan Zhang, Ann Arbor, MI (US)

(72) Inventors: Songtao Wu, Ann Arbor, MI (US); Hongfei Jia, Ann Arbor, MI (US); Masahiko Ishii, Okazaki City (JP); Minjuan Zhang, Ann Arbor, MI (US)

(73) Assignees: Toyota Motor Corporation, Toyota (JP); Toyota Motor Engineering & Manufacturing North America, Inc., Erlanger, KY (US)

(21) Appl. No.: 13/529,322

(22) Filed: Jun. 21, 2012

(51) Int. Cl. A61L 15/46; C12N 11/04; C12N 11/18; A61L 15/32

(52) U.S. Cl. 604/359; 435/182; 435/175

(57) ABSTRACT

A sanitary article is provided that includes a dried polymeric matrix with embedded enzyme. The presence of the enzyme in the polymeric matrix allows the sanitary article to absorb a bodily discharge fluid thereby allowing the embedded enzyme to function in the production of antibacterial compounds or by directly reducing the presence of viable bacteria in the discharge fluid. The enzyme activity promotes reduced odor in the sanitary article.

### FIELD OF THE INVENTION

The invention relates to the field of materials for deodorizing applications. More specifically, the invention relates to materials suitable for deodorization application in sanitary items such as diapers or feminine napkins.

### BACKGROUND OF THE INVENTION

It has long been recognized that most of the human odor that accompanies perspiration or discharge fluids is caused by the action of bacteria and specific enzymes released by these bacteria. Typical mechanisms for removing odor involve abating odor components involve simply masking an unpleasant odor with a pleasant odor. These mechanisms are found lacking and are difficult to achieve in a closed environment such as the interior of a diaper or feminine napkin when being worn.

As such, new materials are needed to provide stable, viable, and active mechanisms for reducing, preventing or eliminating odor in sanitary articles.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A sanitary article and processes for reducing odor within a sanitary article are provided. Odor control is achieved in a sanitary article by including a polymer matrix housing one or more enzymes that are operable to control odor by one or more processes including formation of a compound with antibacterial properties, direct action on bacteria to reduce the bacterial prevalence or alter bacterial activity, to degrade or otherwise alter an odor compound itself, or combinations thereof. One or more of these mechanisms, optionally combined with the ability of the polymer matrix to form a hydrogel capable of absorbing odor compounds, provides a synergistic mechanism for reducing, preventing, or eliminating odor in a sanitary article. In some embodiments, the combined activity of glucose oxidase, α-amylase, and γ-amylase, serve to convert common sugars to antibacterial agents at the site it is most needed—in a sanitary article. The dried enzyme storage medium provided allows for excellent enzyme stability and activation when contacting an aqueous discharge fluid.

Processes for reducing odor from a discharge fluid are provided where a sanitary article with an enzyme storage medium are formed or used to reduce odor from a discharge fluid.

### BRIEF DESCRIPTION OF THE DRAWINGS

FIG. 1 illustrates the size distribution of particles of polymer matrix with entrapped enzyme according to one embodiment of the invention;

FIG. 2 illustrates release of glucose oxidase from swollen enzyme storage medium over time according to one embodiment of the invention;

FIG. 3 illustrates the ability of enzyme included in an enzyme storage medium to produce hydrogen peroxide;

FIG. 4 illustrates the ability of one embodiment of a dried enzyme storage medium to maintain enzyme activity over time and at elevated temperature.

### DETAILED DESCRIPTION OF EMBODIMENTS OF THE INVENTION

The following description of particular embodiments is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only, unless otherwise indicated. While processes and compositions may be described as an order of individual steps or using specific materials, it is appreciated that described steps or materials may be interchangeable such that the description of the invention includes multiple parts or steps arranged in many ways as is readily appreciated by one of skill in the art.

A primary mechanism for enzymatic deodorization is by prevention or inhibition of bacterial growth or bacterial enzyme release, thereby abating odor components produced by bacterial enzymatic action. While enzymes have been used in cleaning compositions, due to delicate enzyme structure necessary for activity, enzyme stability presents a serious hurdle when incorporated into the products such as diapers or sanitary napkins that must be prepared and initially used in a dry format. The inventors have discovered that a dry a polymer matrix including particular enzymes and combinations of enzymes is capable of maintaining enzyme function for use upon swelling and forming a hydrogel. The enzymes associated with the polymer matrix are, therefore, available to actively reduce or prevent odor in a sanitary article. As such, the invention has utility for the reduction or prevention of odor in a sanitary article. Compositions and methods are provided that improve the effectiveness of deodorants in sanitary products. Compositions include one or more enzymes that are entrapped in a polymer matrix, optionally together with fragrant and other antibiotic reagent. In some embodiments, the polymer matrix is dried and optionally reduced in size, protecting the enzymes in the gel matrix due to a confinement effect. Upon contact with an aqueous fluid, the polymer matrix swells to form a hydrogel allowing function of the entrapped enzymes to prevent or reduce the presence of compounds recognized by a human as an odor.

A sanitary article is provided that includes a first enzyme in a polymer matrix to form an enzyme storage medium. The enzyme storage medium is included in a sanitary article. A sanitary article, as defined herein, is an article meant to be worn by a user or contact an epidermal layer of a user whether external or internal and is localized so as to capture or contact one or more discharge fluids. Illustrative examples of sanitary articles and methods of their production are illustratively described in U.S. Pat. Nos. 3,563,242; 3,875,943; 4,534,769; 5,466,232; 6,700,034; 7,132,479; and U.S. Patent Application Publication No: 2012/0089111. A discharge fluid is illustratively sweat, urine, feces, blood (excluding venous or arterial blood), or one or more components thereof. The term “sanitary” is defined herein as substantially free of material or organisms normally considered to be undesirable when contacted with the skin of a human. A sanitary article may be, but need not necessarily be, sterile. A sanitary article is not a means of first aid or intended for medical use for a condition other than incontinence. Illustrative examples of a sanitary article include a diaper, feminine napkin, tampon, lining for an animal housing unit, or other item expected to contact one or more discharge fluids. As used herein a diaper is intended to include articles to be used by infants, as well as children and adults as an aid for incontinence. The invention shall be described herein with respect to a diaper or feminine napkin, but it is appreciated that other sanitary articles are similarly within the scope of the invention.

A sanitary article includes a polymer matrix with an enzyme entrapped within the polymer matrix. An enzyme is optionally covalently associated with one or more components of a matrix, or is non-covalently associated with a matrix. Covalent interactions are optionally mediated by free amino or carboxyl groups on a terminus or an amino acid side chain of an enzyme. In preferred embodiments, an enzyme is non-covalently associated with a polymer matrix such as by entrapment during production of the polymer matrix in the presence of the enzyme at a desired concentration.

An enzyme is optionally any enzyme suitable for the prevention of bacterial growth, or for lowering the amount of odor molecules. An odor molecule is one or more volatile chemical compounds that humans or other animals perceive by the sense of olfaction. The widest range of odor molecules consists of organic compounds (like esters, linear terpenes, cyclic terpenes, aromatics, alcohols, aldehydes, ketones), although some inorganic substances, such as hydrogen sulfide and ammonia, are also odor molecules. Specific examples of odor molecules include nitrogen containing compounds, illustratively ammonia. In particular embodiments, the sanitary article includes one or more enzymes that decrease the viability or presence of one or more species of bacteria. Bacteria are known to produce odor molecules by the action of bacterial urease, which catalyzes the hydrolysis of urea into carbon dioxide and ammonia. Thus, by preventing the presence, viability, or activity of bacteria or enzymes produced by bacteria, odor is controlled.

The choice of the enzyme to include in a polymer matrix depends on the required properties of the polymer matrix. Enzymes and active fragments of enzymes in non-limiting examples include: a glucose oxidase; a glycoside hydrolase, illustratively α-, β-, γ-amylase, lysozyme, or combinations thereof; a free-radical detoxifying enzyme illustratively superoxide dismutase and SOD enzyme mimics; enzymes with proteolytic activity, such as collagenase, trypsin, chymotrypsin, elastase, and/or combinations thereof; protease inhibitors from the class of tissue inhibitors of matrix metalloproteinases (TIMPs) and/or other proteinogenic protease inhibitors such as aprotinin, soy bean trypsin inhibitor and alpha-2 macroglobulin; enzymes with antimicrobial activity, such as, for example, lysozyme and hydrolase; enzymes with phosphorylating activity, such as phosphatases and kinases, growth factors such as, for example, PDGF. In some embodiments, a polymer matrix does not include a protease. Optionally, proteases are absent and a protease inhibitor is present in the polymer matrix. In some embodiments, the active derivatives such as fragments of enzymes that maintain some degree of enzymatic activity relative to the wild-type are encompassed under the term enzymes. Optionally, an enzyme specifically excludes derivatives of wild-type enzymes. An enzyme is present at a concentration that will allow for enzymatic activity to be detectable. Optionally, an enzyme is present at a level from 0.1 to 5% (w/w) or any value or range therebetween. Optionally, an enzyme is present from 0.1 to 1% (w/w), optionally from 0.2% to 0.5% (w/w).

An enzyme for controlling odor and inclusion in a sanitary article in particular embodiments includes: glucose oxidase; an amylase, lysozyme; or combinations thereof. In some embodiments, a polymer matrix includes a first enzyme. A first enzyme is optionally an amylase, or glucose oxidase. Optionally, a first enzyme is glucose oxidase. In some embodiments, more than one enzyme is present in a single polymer matrix, or more than one polymer matrix is present with individual or separate enzyme types. In some embodiments, two enzymes are present. Optionally, two or more enzymes are present. In embodiments with two or more enzymes, a first enzyme is optionally glucose oxidase, and a second enzyme is an amylase. Optionally, three enzymes are present in a polymer matrix. In some embodiments, a polymer matrix includes glucose oxidase, α-amylase, and γ-amylase.

Controlling odor in a sanitary article is optionally a multi-step process. Several bodily fluids illustratively, urine or blood, include sugar molecules that are formed from glucose in whole or in part. The presence of one or more amylase enzymes will cause the breakdown of complex sugars to constituent parts including glucose. Glucose oxidase will then act on the glucose product in the presence of the water and oxygen to produce D-gluconic acid and hydrogen peroxide. The hydrogen peroxide has antibacterial activity such that its production by an enzyme storage medium will lead to antibacterial activity in the sanitary article. The resulting reduction in bacterial activity or propensity will lead to elimination, reduction, or prevention of odor in the sanitary article. As such, some embodiments capitalize on a synergistic relationship between amylases and glucose oxidase present in a single or plurality of polymeric matrices to reduce or eliminate odor in a sanitary article.

Anti-bacterial activity is achieved in some embodiments by production of general bactericide H₂O₂ from sugar molecules in the discharge fluid using a combination of glucose oxidase and amylase in a single polymeric matrix or by intermixing of a plurality of polymeric matrices with different enzyme constituents, by elimination of bacteria itself, inhibition of enzymes released by bacteria, or combinations thereof. A polymeric matrix optionally includes an antibacterial enzyme. Illustrative enzymes that have antibacterial activity include, but are not limited to lysozyme and glycosidases, cationic proteins including low molecular weight proteins, and lactoperoxidase. The presence of lactoperoxidase in a polymer matrix forms a synergistic relationship with glucose oxidase. In the presence of hydrogen peroxide produced by the glucose oxidase, lactoperoxidase can disrupt bacterial cell membranes resulting in cell death. Other antibiotic reagents can also be introduced to strengthen the inhibition effects. Illustrative examples of antibiotic reagents include quaternary ammonium compounds, phenols, amides, acids and nitro compounds, and combinations thereof. Specific examples of antibiotic reagents include those listed in U.S. Pat. No. 7,132,479.

A fragrant is optionally included in an enzyme storage medium or elsewhere in a sanitary article to mask odor from odor compounds. Illustrative examples of odor masking substances include deodorants and perfumes. Specific examples of a perfume are found in U.S. Pat. No. 7,132,479 and illustratively include allyl caproate, allylcyclohexane acetate, allylcyclohexane propionate, allyl heptanoate, amyl acetate, amyl propionate, anetole, anisole, benzaldehyde, benzyl acetate, benzylacetone, benzyl alcohol, benzyl butyrate, benzyl formate, benzyl isovalerate, benzyl propionate, butyl benzoate, butyl caproate, camphor, cis-3-hexenyl acetate, cis-3-hexenyl butyrate, cis-3-hexenyl caproate, cis-3-hexenyl valerate, citronellol, citronellyl derivatives, Cyclal C, cyclohexylethyl acetate, 2-decenal, decylaldehyde, dihydromyrcenol, dimethylbenzylcarbinol and derivatives thereof, dimethyloctanol, diphenyl oxide, ethyl acetate, ethyl acetoacetate, ethyl amyl ketone, ethyl benzoate, ethyl butyrate, ethyl hexyl ketone, ethyl phenylacetate, eucalyptol, fenchyl acetate, fenchyl alcohol, tricyclodecenyl acetate, tricyclodecenyl propionate, geraniol, geranyl derivatives, heptyl acetate, heptyl isobutyrate, heptyl propionate, hexenol, hexenyl acetate, hexenyl isobutyrate, hexyl acetate, hexyl formate, hexyl isobutyrate, hexyl isovalerate, hexyl neopentanoate, hydroxycitronellal, α-ionone, β-ionone, γ-ionone, isoamyl alcohol, isobornyl acetate, isobornyl propionate, isobutyl benzoate, isobutyl caproate, isononyl acetate, isononyl alcohol, isomenthol, isomenthone, isononyl acetate, isopulegol, isopulegyl acetate, isoquinoline, dodecanal, lavandulyl acetate, ligustral, δ-limonene, linalool and derivatives, menthone, menthyl acetate, methylacetophenone, methyl amyl ketone, methyl anthranilate, methyl benzoate, methyl benzylacetate, methylchavicol, methyleugenol, methylheptenone, methyl heptynecarbonate, methyl heptyl ketone, methyl hexyl ketone, methylnonylacetaldehyde, α-iso“γ”methylionone, methyloctylacetaldehyde, methyl octyl ketone, methylphenylcarbinyl acetate, methyl salicylate, myrcene, myrcenyl acetate, neral, nerol, neryl acetate, nonalactone, nonyl butyrate, nonyl alcohol, nonyl acetate, nonylaldehyde, octalactone, octyl acetate, octyl alcohol, octylaldehyde, D-limonene, p-cresol, p-cresyl methyl ether, p-cymene, p-isopropyl-p-methylacetophenone, phenethyl anthranilate, phenoxyethanol, phenylacetaldehyde, phenylethyl acetate, phenylethyl alcohol, phenylethyldimethylcarbinol, α-pinene, β-pinene, α-terpinene, γ-terpinene, terpineol, terpinyl acetate, terpinyl propionate, tetrahydrolinalool, tetrahydromyrcenol, thymol, prenyl acetate, propyl butyrate, pulegone, safrole, δ-undecalactone, γ-undecalactone, undecanal, undecyl alcohol, veratrol, verdox, vertenex, viridine, or combinations thereof. Perfumes may be either in free form or complexed in a cyclodextrin envelope.

It is appreciated that a sanitary article optionally includes molecules that will complex with odor compounds such as small molecules or metal ions. Cyclodextrins are suitable for such a purpose. Illustrative examples of cyclodextrins and combinations of cyclodextrins are illustratively found in U.S. Pat. No. 7,132,479.

Hydrated polymer matrix (i.e. hydrogel) can also absorb malodor compounds within its porous structure. By applying multiple odor control mechanisms together in a single polymer matrix, greater deodorant activity can be expected.

One or more enzymes are present in a single polymer matrix. A polymer matrix is a material formed of polymers known as superabsorbent polymers. Super absorbent polymers are interconnected networks of hydrophilic molecules that are capable of swelling in the presence of water and shrinking during a dehydration process. In this way, a dried polymer matrix can absorb large amounts of liquid thereby forming a hydrogel that will hold the liquid in the polymer matrix. These superabsorbent polymers find excellent applications in sanitary articles such as diapers and feminine napkins. As such, a sanitary article includes as an enzyme storage medium a dehydrated polymer matrix formed of superabsorbent polymers complexed with or entrapping one or more enzymes. The inclusion of dehydrated polymer matrix allows for absorption of aqueous fluid when contacted with a sanitary article such that substrates and cofactors are drawn in contact with an enzyme. By swelling, the resulting hydrogel, in some embodiments, can then release some amount of enzyme to act at regions adjacent to the hydrogel. The term “hydrogel” as used herein refers to a swelled or water binding polymer matrix of superabsorbent polymers that possess greater than 20% water saturation.

A suitable polymer matrix includes macromolecular and polymeric materials into which water and small molecules can easily diffuse. A polymer matrix is prepared through cross-linking of suitable monomers, where cross-linking may be either through covalent, ionic or hydrophobic bonds introduced through use of either chemical cross-linking agents or electromagnetic radiation, such as ultraviolet light. A polymeric matrix may include one or more of natural or synthetic hydrophilic polymers, including homo and hetero-polymers. A polymeric matrix is optionally prepared through the cross-linking of olefinically unsaturated carboxylic acids or derivatives thereof with copolymers of C₂-C₈ olefins or derivatives thereof, or styrenes with anhydrides. Specific examples of precursors for polymerization include: ethylene; propylene; isobutylene; 1-butylene; C₁-C₄-methacrylates, vinyl acetate; methyl vinyl ether; isobutyl vinyl ether; 1-hexene; acrylamides; among many others known in the art. Specific examples superabsorbent polymers are described in U.S. Pat. Nos. 5,626,863; 5,573,934; 5,567,435; 5,529,914; 5,514,380; 5,476,909; 5,041,292; 6,773,703; 7,731,479, and 7,605,232. Although the examples provided herein are directed to a polyacrylamide polymer matrix, it is appreciated that other polymer matrix materials known in the art are similarly suitable and one of skill in the art understands both where such materials and precursors may be obtained and how such materials may be employed.

A polymer is optionally cross-linked with one or more cross-linkers suitable for cross-linking the particular polymeric material. A crosslinker includes at least two regions capable of reacting with a polymeric structure, or portion thereof, to chemically link two or more polymeric structures. Illustrative examples of cross-linkers include molecules containing two or more unsaturated bonds, molecules with at least one polymerizable ethylenically unsaturated group and at least one additional functional group; or a molecule with two or more functional groups. Illustrative examples of a functional group include hydroxyl, amino, epoxy, isocyano, ester, and amido groups. Illustrative examples of a crosslinker include N,N′-methylenebisacrylamide, ethoxylated trimethylolpropane triacrylate (ETMPTA), polyethylene glycol diacrylates and polyethylene glycol dimethacrylates, polyhydric alcohols, pentaerythritol triallyl ether, divinylurea, ethylene glycol, diethylene glycol, triethylene glycol, tetraethylene glycol, polyethylene glycol, glycerol, sorbitol, starch, and others listed in U.S. Pat. No. 7,132,479. In addition, multivalent metal anions such as magnesium, calcium, barium and aluminum ions may be used as a crosslinker.

Physical characteristics such as size, shape and surface area can affect the absorption characteristics of the polymer matrix composition. The polymer matrix may be in a variety of configurations, including particles, beads, rods, sheets, irregular shapes, among others. In some embodiments, those shapes with lower surface area to volume ratios are used in total or in major proportion. For example, in some embodiments spheres, irregular spheres, ellipsoid, or other low surface area to total mass ratio shapes are used. The configuration of the polymer matrix may be related to the particular method of production, to the components of the polymer matrix, or both. For example, one method of producing an enzyme containing polymer matrix is by polymerization in an inert solvent. Illustrative examples of an inert solvent include acetone, DMSO, dioxane, ethyl acetate, chloroform, and toluene. In some embodiments, toluene is used in the production of particles of enzyme containing polymer matrix.

The porosity of the polymer matrix is dependent on the amount or degree of cross-linking. Porosity also affects the absorption characteristics of the polymer matrix as well as the ability to retain or secrete an entrapped enzyme upon hydration of the polymer matrix. In some embodiments, the polymer matrix is of suitably high porosity that one or more enzymes are capable of migrating out of the polymer matrix when the matrix is hydrated. Optionally, the polymer matrix is of suitably low porosity that all enzymes will remain entrapped in the polymer matrix when the matrix is hydrated. The porosity is optionally tailored to select for enzymes that are able to migrate from the polymer matrix when hydrated. For example, in some embodiments a polymer matrix is tailored such that glucose oxidase remains trapped in the polymer matrix, but amylases are able to migrate through and possibly outside the polymer matrix. In some embodiments, a polymer matrix is tailored such that all enzymes entrapped in the polymer matrix may migrate from the matrix to act on substrate molecules both within and outside the polymer matrix. In some examples, a polymer matrix is formed from acrylamide cross-linked with bis-acrylamide wherein the acrylamide concentration is 19% or less, optionally 0.1 to 20% or any value or range therebetween; optionally 0.1 to 10%, optionally 1 to 5%. This will allow large enzymes such as glucose oxidase with a molecular weight of 160,000 Da to migrate from the polymer matrix when swelled. The rate of enzyme release is related to the porosity of the polymer matrix. More rapid enzyme migration will occur with greater porosity (lower polymer density). One of skill in the art readily understands how to produce a polymer matrix with a desired porosity by adjusting the relative amounts of polymerization precursors. Illustrative examples of methods of producing a polymer matrix are found in U.S. Pat. No. 7,132,479.

A polymer matrix is optionally in the form of a plurality of particles that are substantially spherical, elongated, rod shaped, irregularly shaped, or combinations thereof. In some embodiments, a particle has a linear dimension, optionally diameter, of 1 millimeter or less when dehydrated. A linear dehydrated dimension is optionally 100 nm to 100 μm or any value or range therebetween. Optionally, an average linear dehydrated dimension is 0.5 μm to 5 μm, 0.5 μm to 2 optionally about 1 μm. The term “about” is intended to mean within experimental or production error, optionally 10%. A plurality of particles is not necessarily uniform in size or shape. The inventors have discovered that particle size is important to promoting migration of an entrapped enzyme into the extraparticulate space thereby improving the odor control of the system. As such, improved action is observed in particles with a linear dehydrated dimension of 0.5 μm to 5 with further improvements found using an average linear dehydrated dimension of 1 μm or less. Excellent results are observed with particles that are substantially circular with a diameter of 1 μm or less.

An enzyme storage medium is associated with all or a portion of a sanitary article. In some embodiments, an enzyme storage medium is dispersed throughout a sanitary article. In some embodiments, an enzyme storage medium is located in one or more discrete or particular regions of a sanitary article. A discrete or particular region is one that is expected to contact one or more discharge fluids when the sanitary article is used. In some embodiments, a sanitary article is a diaper or a feminine napkin. A diaper or feminine napkin optionally includes an enzyme storage medium in a position such that the enzyme storage medium is not exposed to the exterior of the sanitary article. Illustratively, an enzyme storage medium is sandwiched between a water permeable inner layer and an outer layer, optionally water impermeable, where the inner layer is intended to be proximal to the epidermis of a subject when wearing or using a sanitary article. This allows one or more discharge fluids to penetrate an inner layer and swell the enzyme storage medium thereby providing active enzyme capable of reducing, preventing, or eliminating odor compounds or odor producing bacteria. An inner layer is optionally made of any material suitable for contacting the epidermis of a wearer. Illustratively, an inner layer is synthetic or manufactured fibers or films of polyesters, polyolefins, rayon or natural fibers such as cotton. An outer layer is optionally any water impervious material, illustratively, polyethylene or polypropylene.

Methods of sanitary article construction are known in the art. Illustratively, diaper construction is exemplified in WO 95/26209 page 66 line 34 to page 69 line 11. Feminine napkin construction is illustratively taught in WO 95/24173. Articles for use in incontinence are illustratively described by WO 95/26207. The enzyme storage medium may be included in any of the aforementioned articles and constructions, but are appreciated to be applicable to numerous other designs and constructions as are known to one of skill in the art.

An enzyme storage medium is may be formed by one of several methods known in the art. Illustratively, the polymers used to form the polymeric matrix are prepared by free-radical polymerization in an aqueous solution which includes the monomers, one or more enzymes and also, if appropriate, one or more cross-linkers. Illustrative examples of methods useful for forming a polymer matrix are illustrated in U.S. Pat. No. 7,132,479. Polymerization is initiated by the addition of an initiator or by the action of high energy radiation in the presence of one or more photoinitiators. An initiator illustratively includes peroxides, hydroperoxides, hydrogen peroxides, persulfates, azo compounds, redox catalysts, or combinations thereof, among others known in the art. Illustratively, a combination of ammonium persulfate and tetramethylethylenediamine are used. Other suitable polymerization inhibitors known in the art are also useful. The polymerization initiators are included in a polymerization reaction in amounts understood in the art, illustratively, from 0.01 to 5%, based on the monomers to be polymerized.

Typical polymerization reactions include the polymerization of 0.1 to 20% of a solution of monomer to be polymerized along with one or more enzymes at an effective amount. In preferred embodiments, the amount of monomer is low enough so as to produce a hydrogel that will allow migration of one or more enzymes away from the hydrogel core. An effective amount is typically formed by the inclusion of micromolar amounts of enzyme in a polymerization reaction. Illustratively, glucose oxidase or an amylase is included from 1 micromolar to 1 millimolar.

The polymerization reaction can be performed at a temperature where the solvent used will remain liquid. Illustrative temperatures include 0° C. to 150° C. Preferred temperatures are less than 70° C., more preferably less than 55° C.

An atmosphere is illustratively air, or a protective atmosphere such as nitrogen. Standard atmospheric pressure is typically used, but higher or lower pressure environments are similarly suitable depending on the exact reaction conditions being employed.

Materials for the formation of an enzyme storage medium including the procures for formation of a polymeric matrix are found from many commercial sources known in the art, illustratively, Sigma-Aldrich, Co., St. Louis, Mo.

Methods for formation of a polymer matrix include all processes which are customarily used to make superabsorbents, but also include the addition of one or more enzymes in the polymerization reaction. Illustrative examples of methods for making a polymer matrix of superabsorbents are described in Chapter 3 of “Modern Superabsorbent Polymer Technology”, F. L. Buchholz and A. T. Graham, Wiley-VCH, 1998, as well as in U.S. Pat. No. 7,132,479.

Also provided are processes of reducing, preventing, or eliminating odor in a sanitary article prior or subsequent to a sanitary article contacting a discharge fluid. A process includes providing an enzyme housed in a polymer matrix to form an enzyme storage medium and associating the enzyme storage medium with a sanitary article, or portion thereof, such that the enzyme will be active when the sanitary article contacts an aqueous fluid. An enzyme storage medium will be associated with a sanitary article so that an enzyme is active when the sanitary article contacts an aqueous fluid when the enzyme storage medium is positioned within, on, or both, the sanitary article so that contact on a surface of a sanitary article will allow discharge fluid to contact the enzyme storage medium or penetrate all or a portion of the sanitary article so that enzyme storage medium contained in the sanitary article will be in contact with the discharge fluid or a portion thereof. In the processes, any sanitary article according to the invention described herein, and equivalents, is operable in one or more embodiments.

A process optionally includes contacting a sanitary article with one or more biological fluids, allowing the enzyme storage medium to form a hydrogel, and reducing, preventing, or eliminating odor by the contact with the aqueous medium.

In some embodiments of a process, a sanitary article includes two or more enzymes within a polymer matrix. Optionally, two or more amylases are included in combination with glucose oxidase. Optionally, a process includes forming, providing, or obtaining an enzyme housed in a polymer matrix to form an enzyme storage medium. Optionally, α-amylase, γ-amylase, and glucose oxidase are included in a single or multiple polymeric matrices.

Various aspects of the present invention are illustrated by the following non-limiting examples. The examples are for illustrative purposes and are not a limitation on any practice of the present invention. It will be understood that variations and modifications can be made without departing from the spirit and scope of the invention. Where to obtain or how to produce reagents and materials illustrated herein is readily understood by a person of ordinary skill in the art.

### Example 1

An enzyme storage medium is formed by the polymerization of monomers or shorter chain polymers in the presence of a desired amount of one or more enzymes. Prepolymers of acrylamide are combined with bis-acrylamide in solution (1.2 ml) and mixed with 100 μl of fresh prepared 10% w/v ammonium persulfate and 2.5 mg of glucose oxidase and α-amylase (equal amounts) in water. The solution is loaded into a syringe. A small amount of tetramethylethylenediamine (TEMED) (10 μl) and 4 g of sodium dioctyl sulfosuccinate (AOT) surfactant are added into 40 ml of toluene in a beaker with stirring at 500 rpm for 30 minutes. The enzyme mixture in the syringe is then pumped via a syringe pump into the toluene solution with stirring at 1100 rpm. Polymer matrix beads are formed under these conditions in about 4 hours. The resulting enzyme storage medium is removed by ultrafiltration and then washed three times with DI water and dried for one week at ambient temperature.

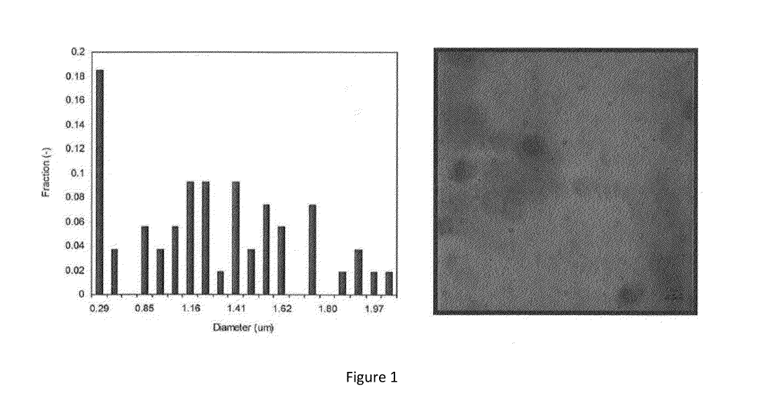

The dried particles of enzyme storage medium with entrapped enzyme are characterized by light microscope. The average size is 1.0 μm (FIG. 1). The beads size can be controlled by adjusting surfactant concentration, stiffing speed, etc.

### Example 2

Enzyme storage medium is formed in the shape of particles by the procedures of Example 1 with glucose oxidase (GOx) (1% w/w) as the sole entrapped enzyme. The beads are swelled in water for 5 minutes prior to enzyme activity assay. A coupled-enzyme reaction is established using horse radish peroxidase and o-dianisidine. For the native GOx, the reaction mixture (1.1 ml) contains 0.1 g glucose, 7 μg horseradish peroxidase, 0.17 mM o-dianisidine, and 35 μl enzyme (0.4-0.8 unit/ml) in 50 mM sodium acetate buffer pH 5.1. The increase in absorbance at 500 nm at room temperature is recorded and compared to a standard curve to determine enzyme activity. The reaction with hydrogel-entrapped GOx is performed in 20-ml vials. Excellent enzyme activity is measured in the beads incorporating GOx as an enzyme.

### Example 3

An enzyme storage medium is formed by the procedures of Example 1 with the inclusion of chymotrypsin (1% w/w). The beads are swelled in water for 5 minutes prior to enzyme activity assay. A standard curve is established using native chymotrypsin. 50 μl of enzyme solution (either native chymotrypsin or chymotrypsin enzyme storage medium) is mixed with 2.44 ml of Sodium Acetate Buffer (SAB) and 13 μl of 160 mM n-Succinyl-Ala-Ala-Pro-Phe-p-nitroanilide SAAPPN stock solution in a cuvette. The reaction rates are determined by monitoring the absorbance at 410 nm. The swelled beads demonstrate excellent chymotrypsin activity.

### Example 4

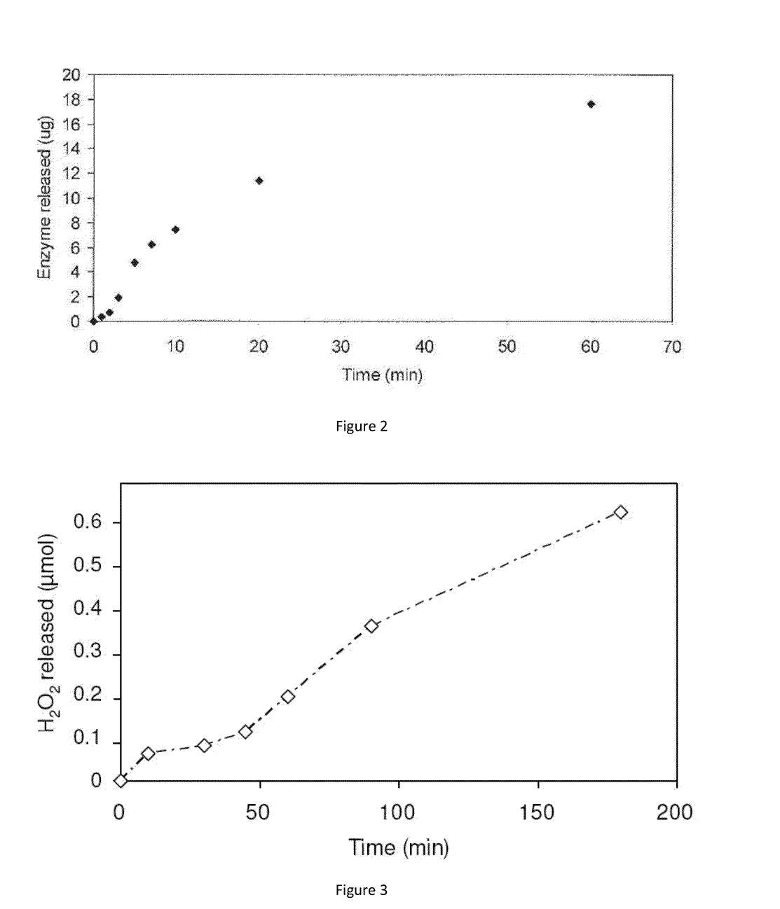

To determine the rate of enzyme release from an enzyme entrapped hydrogel (i.e. swollen enzyme storage medium), beads are prepared by the procedure of Example 1 containing glucose oxidase (1% w/w) are swelled in sodium acetate pH 5.1 for 5 minutes. The amount of enzyme released is measured by Bradford Assay. As is illustrated in FIG. 2, the amount of glucose oxidase released from the hydrogel rapidly rises over the first 10 minutes and continues to rise, albeit more slowly out to the end of the experimental time of 60 minutes.

### Example 5

Swollen enzyme containing hydrogel is active to produce hydrogen peroxide as an anti-odor molecule. Particulate enzyme storage medium is formed by the process of Example 1 with the inclusion of 1% w/w total enzyme (based on dry gel weight): glucose oxidase (0.5%), α-amylase (0.25%), and γ-amylase (0.25%). The amount of hydrogen peroxide produced in the presence of 10 mg (dry weight) of enzyme storage medium is determined by the method of Example 2 replacing starch slurry (3% w/v) for the glucose. The amount of enzyme activity is measured as a relationship to the amount of H₂O₂ produced in the system. As is illustrated in FIG. 3, significant enzyme activity is present in the enzyme containing hydrogel. Enzyme activity remains for over 180 minutes.

### Example 6

To determine direct antibacterial activity of particles of enzyme storage medium including an antibacterial enzyme, a particulate enzyme storage medium is formed by the process of Example 1 using lysozyme (1% w/w) as the entrapped enzyme. To test for antibacterial activity, potassium phosphate buffer (66 mM; pH 6.24) in 100 ml purified water is prepared at 25° C. Micrococcus lysodeikticus (Worthington, Biochemical Co., Lakewood, N.J.) lyophilized cells are suspended in the above buffer at a 0.01% (w/v) solution. Beads (10 mg dry weight) containing lysozyme are swelled in water for 5 minutes prior to enzyme activity assay. Swollen beads are then added into the bacteria solution. Aliquots are taken periodically and placed into a cuvette. The absorbance is determined at 450 nm (A450). One unit is defined as a decrease in A450 of 0.001 per minute at pH 6.24 at 25° C., using 1 cm light path. Excellent antibacterial activity is observed by a reduction A450.

### Example 7

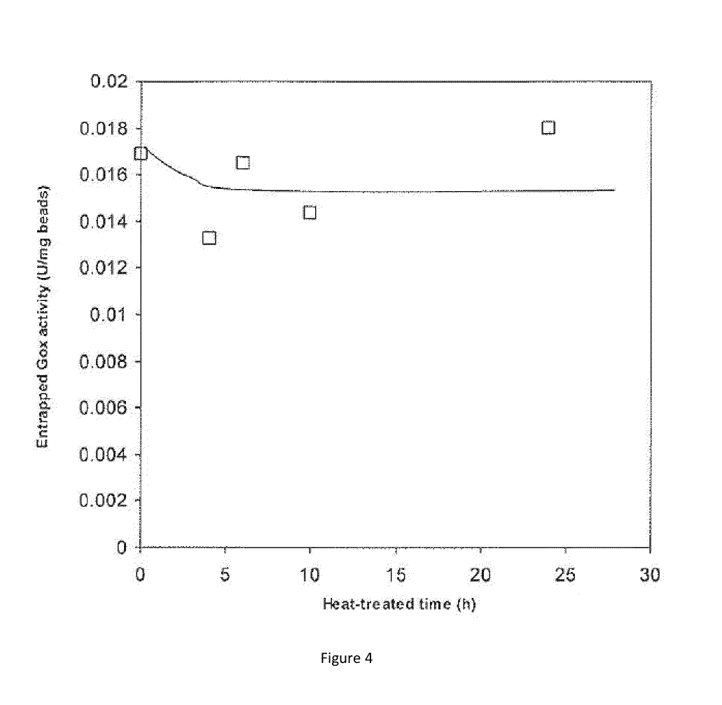

Enzyme present in enzyme dry storage medium has excellent storage capability. Storage capability is determined of a particulate enzyme storage medium prepared as in Example 1 using: glucose oxidase (1%); chymotrypsin (1%); glucose oxidase (0.5%) plus α-amylase (0.5%); lysozyme; or the combination of glucose oxidase (0.4%), α-amylase (0.2%), γ-amylase (0.2%), and lysozyme (0.2%). The resulting particles are then paced in an oven at 80° C. for various time periods after which a sample is taken and individual enzyme activity is determined. The stability of glucose oxidase is maintained in the dried enzyme storage medium for 24 hours at elevated temperature (FIG. 4) demonstrating the excellent stability of an enzyme in this medium. Similar stability levels are observed for α-amylase, γ-amylase, and lysozyme.

### Example 8

Diapers are formed as in U.S. Pat. No. 7,132,479 with the inclusion of enzyme storage medium formed as in Example 1 incorporating either: glucose oxidase (1%); chymotrypsin (1%); glucose oxidase (0.5%) plus α-amylase (0.5%); lysozyme; or the combination of glucose oxidase (0.4%), α-amylase (0.2%), γ-amylase (0.2%), and lysozyme (0.2%). The ability of the a diaper including an enzyme storage medium is performed essentially as described in U.S. Pat. No. 6,277,772 using either a real pooled urine sample or a synthetic urine prepared at the time of use having the following composition: for 1 L of water: urea (25 g), NaCl (9 g), K₂SO₄ (4 g), (NH₄)₂SO₄ (2.5 g), MgSO₄ (0.6 g), glucose (5 g), Ca(OCOCH₃)₂ (0.7 g), and yeast extract (5 g). A test fluid is prepared with 20 mL of real or synthetic urine, 0.5 g of urea, and either with 2 g of soiled cellulose fluff embedded with ammonia odor or with a chosen bacterial strain. The mixture is incubated for 2 days, during which the collected urine is stored at 4° C. At the time of the test, the test fluid has a significant undesirable odor. In the test fluid incorporating isolated bacterial strains, the bacterial concentration is measured and expressed in cfu/mL, in order to provide reproducible and standardized test parameters. Hermetically sealed polyethylene boxes are prepared with one for each product to be tested as well as individual boxes for controls. In each box, a single diaper with or without an enzyme storage medium is deposited. The diapers are baked overnight at 37° C. and presented individually to a blinded test panel. The presence of the enzyme storage medium produces a marked reduction in undesirable odor relative to articles without the enzyme storage medium, or to articles with polymeric matrix absent an enzyme.

### Example 9

Feminine napkins as described in WO 95/24173 are produced with an enzyme storage medium formed by the process of Example 1 incorporating either: glucose oxidase (1%); chymotrypsin (1%); glucose oxidase (0.5%) plus α-amylase (0.5%); lysozyme; or the combination of glucose oxidase (0.4%), α-amylase (0.2%), γ-amylase (0.2%), and lysozyme (0.2%). The feminine napkins are contacted with urine on the interior surface and qualitative assessment of resulting odor by the method of Example 8. The presence of the enzyme storage medium produces a reduction in undesirable odor relative to articles that do not contain enzyme storage medium.

### Example 10

A cellulose (fluff) tampon 6 cm×7.5 cm square is embedded with or without an enzyme storage medium formed as in Example 1 incorporating either: glucose oxidase (1%); chymotrypsin (1%); glucose oxidase (0.5%) plus α-amylase (0.5%); lysozyme; or the combination of glucose oxidase (0.4%), α-amylase (0.2%), γ-amylase (0.2%), and lysozyme (0.2%). The tampon is subjected to the odor reduction test of Example 8. The presence of the enzyme storage medium produces a reduction in undesirable odor.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A sanitary article comprising:
a first enzyme, said enzyme housed in a polymer matrix to form an enzyme storage medium;
said enzyme storage medium included in a sanitary article.

2. The sanitary article of claim 1 wherein said first enzyme is glucose oxidase.

3. The sanitary article of claim 1 wherein said first enzyme is a hydrolase.

4. The sanitary article of claim 1 further comprising a second enzyme.

5. The sanitary article of claim 4 wherein said first enzyme is glucose oxidase and said second enzyme has antibacterial activity.

6. The sanitary article of claim 4 wherein said first enzyme is glucose oxidase and said second enzyme is a glycoside hydrolase.

7. The sanitary article of claim 6 wherein said second enzyme is a mixture of a plurality of glycoside hydrolases.

8. The sanitary article of claim 4 wherein said second enzyme is α-amylase, γ-amylase, or combinations thereof.

9. The sanitary article of claim 1 wherein said enzyme storage medium is in the form of a plurality of particles.

10. The sanitary article of claim 9 wherein said particles have a diameter of 0.1 to 5 micrometers.

11. The sanitary article of claim 9 wherein said plurality of particles has an average particle size of 0.8 to 1.2 micrometers.

12. The sanitary article of claim 1 wherein said sanitary article is a diaper.

13. The sanitary article of claim 1 wherein said sanitary article is a feminine napkin.

14. The sanitary article of claim 1 wherein said sanitary article is a tampon.

15. The sanitary article of claim 1 wherein said enzyme is non-covalently associated with said enzyme storage medium.

16. The sanitary article of claim 1 wherein said polymer matrix comprises acrylamide.

17. The sanitary article of claim 16 wherein the polymer matrix is formed from a percent monomer of acrylamide of at or less than 19 percent.

18. The sanitary article of claim 16 wherein said acrylamide is cross-linked and forms a particle.

19. A process of reducing or preventing odor formation in a sanitary article comprising:
providing a first enzyme, said first enzyme housed in a polymer matrix to form an enzyme storage medium;
associating said enzyme storage medium in a sanitary article, such that said first enzyme is active when said sanitary article contacts an aqueous fluid.

20. The process of claim 19 wherein said first enzyme is glucose oxidase, a glycoside hydrolase, or combinations thereof.

21. The process of claim 19 wherein said polymer matrix further comprises a second enzyme.

22. The process of claim 21 wherein said first enzyme is glucose oxidase and said second enzyme has antibacterial activity.

23. The process of claim 21 wherein said first enzyme is glucose oxidase and said second enzyme is a glycoside hydrolase.

24. The process of claim 23 wherein said second enzyme is a mixture of a plurality of glycoside hydrolases.

25. The process of claim 21 wherein said second enzyme is α-amylase, γ-amylase, or combinations thereof.

26. The process of claim 19 wherein said enzyme storage medium is in the form of a plurality of particles.

27. The process of claim 26 wherein said particles have a diameter of 0.1 to 5 micrometers.

28. The process of claim 26 wherein said plurality of particles has an average particle size of 0.8 to 1.2 micrometers.

29. The process of claim 19 wherein said sanitary article is a diaper, feminine napkin, or tampon.

30. The process of claim 19 wherein said polymer matrix comprises acrylamide.

31. The process of claim 19 wherein the polymer matrix is formed from a percent monomer of acrylamide of at or less than 19 percent.
